(19) United States
(12) Patent Application Publication
(10) Pub. No.: US 2021/0010978 A1
(43) Pub. Date: Jan. 14, 2021

(54) VIBRO-ACOUSTIC DELIVERY SYSTEM

(71) Applicant: Hi Viber, LLC, Bellevue, WA (US)

(72) Inventors: Joel Johanneson, Bellevue, WA (US); Thomas McGrath Lukens, Bellevue, WA (US)

(21) Appl. No.: 16/904,522

(22) Filed: Jun. 17, 2020

Related U.S. Application Data
(60) Provisional application No. 62/873,707, filed on Jul. 12, 2019.

(51) Int. Cl. G01N 29/22; G01N 29/34

(52) U.S. Cl. CPC G01N 29/226; G01N 29/34; G01N 29/221

(57) ABSTRACT

A vibro-acoustic assembly and related systems and methods for applying a wide range of audio frequencies in one or multiple channels to living beings for wellbeing. The assembly is ergonomic, compact, portable and configured to address physically any area of a user's body while being flexible and easy to use to deliver a wide degree of frequencies/identified targeted programs from a source audio device, such as a smart phone application via wireless transmission.

### CROSS REFERENCE TO RELATED APPLICATION

This non-provisional patent application hereby claims the benefit of and priority to U.S. Provisional Patent Application No. 62/873,707, titled Vibro-Acoustic Delivery System, filed Jul. 12, 2019, which is incorporated herein in its entirety by reference thereto.

### TECHNICAL FIELD

The present technology relates generally to vibro-acoustic apparatus and systems adapted to deliver a wide range of high acoustic energy frequencies to a human body, animal body, or plant living structure.

### BACKGROUND

There is an epidemic of overdose and pharmaceuticals in the world where opioids and drugs are the first option for many in the medical field to relieve physical and mental conditions. Over-prescription and misuse of opioids and drugs often lead the users to addictive and undesirable drug treatment effects. In many situations, vibro-acoustic or physio acoustic therapy can be effective for treating patients' physical and mental conditions. Current devices, however, are large and non-portable delivery platforms operated by pre-set and limited function capabilities, tethered to amplifiers and frequency generators, and are unable to specifically physically localize a broad range of mental and physical issues impacting a living being. There is a need for improved vibro-acoustic apparatus, systems, and treatment methods.

### SUMMARY

The present technology provides a vibro-acoustic assembly and related systems and methods that overcome drawbacks of the prior art and provide additional benefits. Embodiments of the present technology provide a vibro-acoustic system for applying a wide range of audio frequencies (Hz) in one or multiple channels to living beings including humans, animals, plants, insects for wellbeing.

The unique ability to control the placement and thus direction of the sound during treatment applied to a body of a patient can stimulate the blood and lymph circulation. In addition, vibro-acoustic assembly is configured to allow for changing of the strength (pulsation) of the sound vibrations so as to prevent overstimulation. The assembly produces audible and sensible sound vibrations for targeted application to the body that will influence the body at a cellular level based on the natural frequency of every human cell. When playing the correct frequency, a specific group of cells will vibrate unconstrained, which is referred to as resonance.

Conventional devices have substantively different constructions and configurations and are inflexible and non-portable, while providing only a fixed set of programs/frequencies that do not physically target specific/localized areas of the user or are physically tethered to signal tuners and amplifiers. In addition, the conventional devices do not provide any means for bio-feedback or real-time self-adjustments. Conventional devices are not portable, are restricted on mobility, and are not able to deliver a significant range/selection of frequencies in multiple channels. Nor do they possess the intelligence to auto calibrate. Because of these litigations in current systems/devices, the access to current systems are limited, expensive, and do not locally target specific areas of the body.

The vibro-acoustic assembly of the present technology is ergonomic, compact, and sized to fits into or onto one's hands, or is wearable. Accordingly, the vibro-acoustic assembly is portable and configured to address physically any area of a user's body while being flexible and easy to use to deliver a wide degree of frequencies/identified targeted programs from a source audio device, such as a smart phone application via wireless transmission.

Compared to the conventional devices, the handheld/wearable vibro-acoustic assembly of the present technology can be applied or placed to any part of a surface/body for more direct transmission of a desired frequency in discrete multiple channels. The technology also provides self/auto calibration combined with the bio-feedback data to provide information on signal variables including amplitude, frequency, and duration. This feedback information can also be used to measure program results to improve the type of programs used within the Vibr™ “library” of programs or tracks for generating the acoustic vibrations at the selected amplitude(s), frequency(ies), and duration(s) for the selected treatment program or protocol.

By affixing or holding the vibro-acoustic assembly of the present technology via hand held directly to the subject, the wireless assembly can be placed anywhere on a body with a wide range of “on demand” frequency programs including multi-channel capability. Amplitude and other variables are auto calibrated and deliver to targeted body areas. (headphones audio combined with separate channels for each of the vibro-acoustic assembly, referred to as VIBR™ devices). By placing the vibro-acoustic assembly localized on the target area, the system of the current technology has the capability of surrounding the target area anywhere on the subject's body and then measuring via bio-feedback, so that the user can get the most effective treatment.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1 is a top isometric view of a vibro-acoustic assembly in accordance with an embodiment of the present technology.

FIG. 2 is a bottom isometric view of a vibro-acoustic assembly in accordance with an embodiment of the present technology.

FIG. 3 is a partially exploded cross-sectional view taken substantially along line 3-3 of FIG. 1.

FIG. 4 is a bottom view of the bottom shell assembly of the vibro-acoustic assembly of FIG. 1.

FIG. 5 is an isometric view of the bottom shell assembly of FIG. 4.

FIG. 6 is a cross-sectional view of the bottom shell assembly of FIG. 5.

FIG. 7 is a top view of the bottom shell assembly of FIG. 5.

FIG. 8 is an isometric cross-sectional view of the bottom shell assembly of FIG. 5.

FIG. 9 is a top view of a top shell of the vibro-acoustic assembly of FIG. 1.

FIGS. 10 and 11 are cross sectional views of the top shell of FIG. 9.

FIG. 12 is a partially exploded view of the top and bottom shells with an EVA disk between the shells.

FIG. 13 is a partially exploded view of the vibro-acoustic assembly of FIG. 1.

FIGS. 14A and 14B are top and side views, respectively, of a structure ring of the vibro-acoustic assembly of FIG. 1.

FIGS. 15A and 15B are top and side views, respectively, of a structure disk of the vibro-acoustic assembly of FIG. 1.

FIG. 16 includes cross sectional and side views of a threaded column of the vibro-acoustic assembly of FIG. 1.

FIG. 17 includes cross sectional and side views of a spacer column of the vibro-acoustic assembly of FIG. 1.

FIGS. 18A and 18B are top and side views, respectively, of a center disk of the vibro-acoustic assembly of FIG. 1, wherein the center disk can be made of a compressed EVA material.

FIGS. 19A and 19B are top and side views, respectively, of a filler ring of the vibro-acoustic assembly of FIG. 1, wherein the filler ring can be made of an EVA material.

FIGS. 20A and 20B are bottom and top isometric views of a push-button switch of the vibro-acoustic assembly of FIG. 1.

FIGS. 21A and 21B are top and side views, respectively, of a flexible molded logo patch of the vibro-acoustic assembly of FIG. 1, wherein the molded logo patch covers the push-button switch of FIGS. 20A and 20B.

FIG. 22 is an enlarged cross-sectional view of one of the support column assemblies located within the vibro-acoustic assembly of FIG. 1.

FIGS. 23-25 are sample screenshots from an App of the present technology for controlling and communicating with the vibro-acoustic assembly of FIG. 1.

Appendix A includes photos of one or more embodiments of the vibro-acoustic assembly of the present technology.

### DETAILED DESCRIPTION

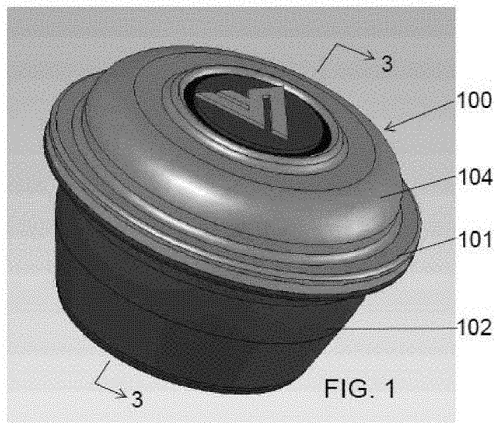

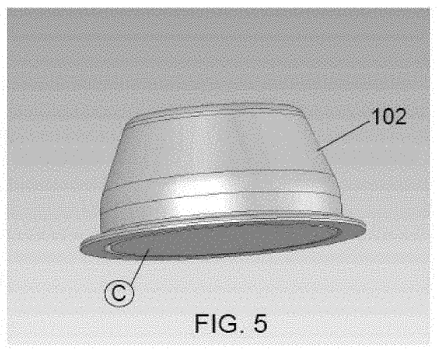

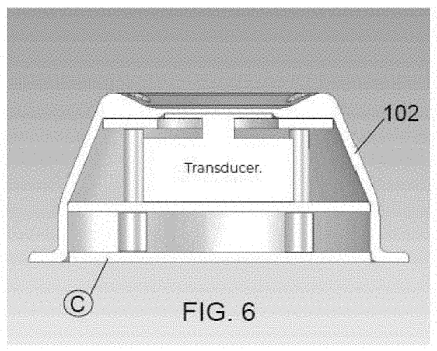

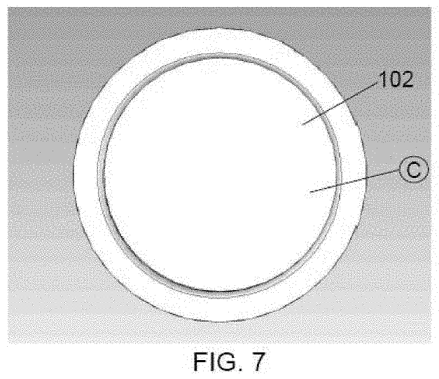

A vibro-acoustic assembly and related components and methods are described in detail herein in accordance with embodiments of the present disclosure. The systems and associated assemblies and/or features overcome drawbacks experienced in the prior art and provide other benefits. Certain details are set forth in the following description and in FIGS. 1-22 to provide a thorough and enabling description of various embodiments of the disclosure. Other details describing well-known structures and components often associated with vibration assemblies and associated with forming such assemblies, however, are not set forth below to avoid unnecessarily obscuring the description of various embodiments of the disclosure. Many of the details, dimensions, angles, relative sizes of components, and/or other features shown in the Figures are merely illustrative of particular embodiments of the disclosure. Accordingly, other embodiments can have other details, dimensions, angles, sizes, and/or features without departing from the spirit and scope of the present disclosure. In addition, further embodiments of the disclosure may be practiced without several of the details described below while still other embodiments of the disclosure may be practiced with additional details and/or features. In the Figures, identical reference numbers identify identical, or at least generally similar, elements. Moreover, one of ordinary skill in the art will appreciate that any relative positional terms such as above, below, over, under, etc., do not necessarily require a specific orientation of the assemblies as described herein. Rather, these or similar terms are intended to describe the relative position of various features of the disclosure described herein.

The terminology used in the description presented below is intended to be interpreted in its broadest reasonable manner, even though it is being used in conjunction with a detailed description of certain specific embodiments of the invention. Certain terms may even be emphasized below; however, any terminology intended to be interpreted in any restricted manner will be overtly and specifically defined as such in this Detailed Description section.

Where applicable, relative terminology such as “about” or “substantially” is used herein as meaning the stated value plus or minus ten percent. References throughout the specification to “one embodiment” or “an embodiment” means that a particular feature, structure, or characteristic described in connection with the embodiment and included in at least one embodiment of the present invention. Thus, the appearances of the phrase “in one embodiment” or “in an embodiment” in various places through the specification are not necessarily all referring to the same embodiments. Furthermore, the particular features, structures, or characteristics may be combined in any suitable manner in one or more embodiments.

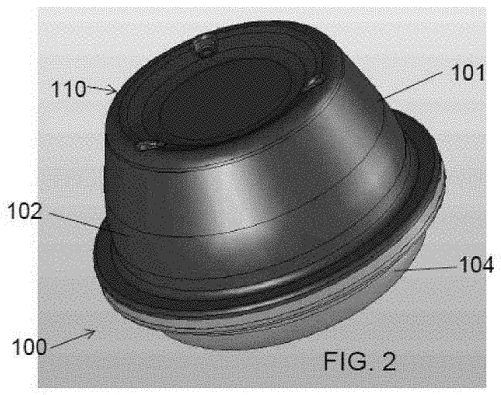

FIGS. 1 and 2 are isometric views of a vibro-acoustic assembly 100 in accordance with an embodiment of the present technology. The vibro-acoustic assembly 100 is adapted to deliver a wide range of high acoustic energy frequencies to a human body, animal body, plant, or other living being. The following discussion will refer to use of the vibro-acoustic assembly 100 with a patient (human or animal), although the description is also applicable for use with other living beings.

A device housing formed by bottom and top shells 102 and 104 coupled together and configured to contain an activatable and controllable transducer to generated selected acoustic vibration for application to a selected location on the patient, such as the patient's body or head. The top shell 104 of the housing can be made of a high-density foam material, and bottom shell 102 can be made of a lower high-density base for transmission of acoustic vibrations therethrough to a targeted location on a patient. The vibro-acoustic assembly 100 with its housing is configured for use via hand to apply the acoustic vibrations to the patient, or the assembly can be used as a “handsfree” standalone system that can be fastened to the patient via straps and pouched straps. For example, hand or “wearable” closure straps or custom straps (i.e., neoprene strap) can be used for specific body parts (i.e., knee, ankle, etc.). The vibro-acoustic assembly 100 and associated methods are configured for calibrating, measuring, adapting, and applying acoustic frequencies, targeted areas/nonlocal, for producing therapeutic/health/performance effects on the patient.

The vibro-acoustic assembly 100 is configured to provide a delivery system for a multitude of frequencies, such as in the range of approximately 15 Hz-10,000 Hz, in mono, or two channel/stereo channels for targeted application to the patient. The vibro-acoustic assembly 100 provides an ergonomic shape and size for easy and convenient use by or on a patient. The hard foam top shell 104 and the lower density foam bottom shell 102 connect together to form a complete hand-holdable unit. The vibro-acoustic assembly 100 can have a two-channel (stereo) body-dual system (Poly-Pulse) which can take advantage of programs that are recorded in stereo to engage the treatment target area from multiple angles. The vibro-acoustic assembly 100 can be used as a stand-alone assembly for applying acoustic vibrations to a patient, or two or more vibro-acoustic assemblies 100 can be calibrated and/or synchronized as left hand and right hand units for use together on the targeted area of the patient. The two or more vibro-acoustic assemblies 100 are paired with each other, so the assemblies can be in communication to each other, and can be self-adjusting and/or co-resonating to provide the desired sound and tactile/vibration. Integrated transducers with sensors from each unit, when used in a pair, provide the program the information necessary to self-adjust variables such as vibration amplitude and frequency. The vibro-acoustic assemblies 100 can include one or more re-chargeable batteries that provide 50% continuous volume for 6+ hours.

The vibro-acoustic assembly 100 and its internal transducer is configured for wireless connectivity with an external controller, such as a smart phone, tablet, or other controller and/or App to apply a program or track of acoustic vibration. The system including the external controller can include a library of frequencies, programs, and/or tracks. In one embodiment, an on-line library or an on-line store can provide a continuous supply of “packaged” programs, which can be targeted for selected treatment or treatment regiments for a patient or selected body part. In some embodiments, vibro-acoustic assembly 100 can be configured for use with wireless and/or wired audio headphones. The assembly can receive and play the vibro-acoustic programs or tracks that provide a human-detectible audio stream (e.g., music) concurrent with the acoustic vibration treatment program or track for the vibro-acoustic treatment of the selected body part. For such embodiments, the vibro-acoustic assembly 100 has a controller and PCB configured to connect and pair one or more vibro-acoustic assemblies 100 with each other via, as an example, a Bluetooth arrangement, and/or with audio headphones into the same audio stream, to which a user or operator can listen. The audio stream may include oral instructions, guidance, or treatment suggestions, and/or the audio stream can provide music, background noise, or other audio content for the user during application of the vibro-acoustic treatment. In other embodiments, the system can provide independent or synchronized user programs to provide, for example, the healing/balancing/harmonizing of various physical and emotional issues, human, animal (large or small) muscle/tissue/bone, insects, treatment, plant treatment, or general wellbeing. The vibro-acoustic assembly and associated systems and methods specifically deliver in a targeted manner the acoustic frequencies to a living being for producing therapeutic/health/performance effects without the need for adhesives or pharmaceuticals.

The system and vibro-acoustic assembly 100 are adaptable for use with chosen frequencies for any part of the patient's body/skin, while maintaining complete flexibility due to its size and mobility with no external wires or cables. The same vibro-acoustic assembly 100 can be used for some or all treatment and/or audio programs. In other embodiments, one set of vibro-acoustic assemblies 100 can be specifically configured for some body parts or treatment protocols, while one or more other sets of vibro-acoustic assemblies 100 can be configured for other body parts and/or treatment protocols. The vibro-acoustic assembly 100 can include an integrated DAC (digital-analog converter) that utilizes digital signals or programs, while delivering analog signals for the treatment.

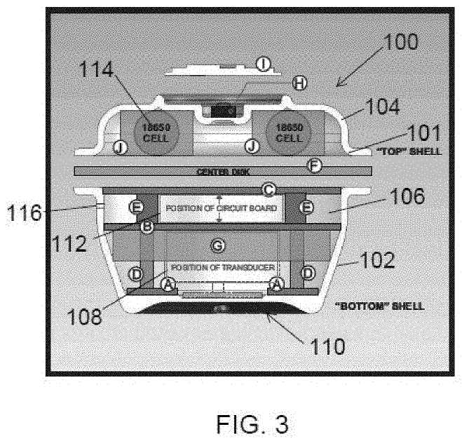

As seen in FIGS. 1 and 2, the vibro-acoustic assembly 100 of the present technology has the housing unit 101 formed by the molded top and bottom shells 104 and 102. A housing 101 forms a handheld unit that can be held and applied to selected portions of the patient's body. The housing 101 can also include adjustable straps that allow the assembly to be temporarily attached to the patient's body and the selected location for use in a wearable, hands-free manner. As seen in FIG. 3, the top and bottom shells 104 and 102 define an interior area 106 that contain the assembly's internal components, including a transducer 108 that is controllable and operable to generate vibro-acoustical signals (i.e., mechanical vibration) with a frequency in the range of approximately 15 Hz-10,000 Hz. In FIGS. 2 and 3, the bottom shell 102 has a concave design positioned immediately adjacent to the internal transducer, the concave portion 110 faces the patient's body and is configured for focused penetration of the acoustic vibration deep into the targeted cells, muscles, joints, bones, or other targeted area for the selected acoustic therapy. The concave portion is specifically designed with the shape and wall thickness to steer and direct the acoustic waves specifically generated from the transducer 108 in narrow bands to penetrate deeper into the body structure.

As seen in FIG. 3, the bottom shell 102 of the illustrated embodiment is formed of a molded lower density EVA material configured to allow the contoured concave portion 110 to flex and vibrate upon activation of the transducer 108 so as to transmit and direct the vibro-acoustic signals from the transducer through the bottom shell to the selected portion of the patient during treatment. The transducer 108 is affixed to the central area of the concave portion 110, which has a reduced thickness to better facilitate transmission of the signals from the transducer 108 to the patient's body. The bottom shell 102 of the illustrated embodiment contains a structure ring A positioned on the bottom wall of the bottom shell 102. The structure ring A has an open central area that receives a portion of the transducer assembly 108. A middle structure disk B is above and spaced apart from the structure ring A above the transducer assembly 108. And upper structure disk C is positioned above the intermediate structure disk and is generally coplanar with the top of the bottom shell 102. The structure ring A, the intermediate structure disk B, and the upper structure disk C can be made of stiff or generally rigid materials, such as an HDPE sheeting material having a selected thickness.

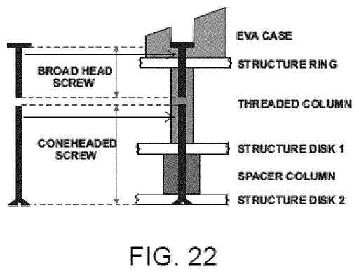

Threaded columns D are affixed to the structure ring A and the intermediate structure disk B to hold them apart and to form the space that receives the transducer 108. A space filler G, which can be made of an EVA, foam, or other suitable material is positioned between the intermediate structure disk B and the structure ring A. The space filler G has a central opening that receives the top portion of the transducer 108 and securely retains the transducer in position within the interior area 106. The upper structure disk C is supported apart from the intermediate structure disk B by spacer columns E so as to provide an area therebetween that contains the circuit board 112. In the illustrated embodiment, the assembly includes a set of three threaded columns D and three spacer columns E joined together with selected fasteners, such as Brodhead screws and/or coneheads screws (see FIG. 22).

In the illustrated embodiment, one or more center discs F are captured between the top and bottom shells 104 and 102 when the housing is fully assembled. The center disk F can have a selected unique color that provides a visual indicator for users, such as in a trademark fashion if desired. The center disk F can be made of a partially compressible material, such as an EVA or foam material that allows for a fixed and solid interconnection between the top and bottom shells 104 and 106. In the illustrated embodiment, the top and bottom shells 104 and 106 to are glued or otherwise adhered to the center disk F to form a permanently closed and substantially sealed interior area. In other embodiments, the top and bottom shells can be joined together in an openable and closable configuration as desired.

The circuit board 112 of the illustrated embodiment includes the amplifier, Bluetooth, DAC, battery charger, indicator, processors, and other control features for operation of the vibro-acoustic assembly. The circuit board 112 and the transducer 108 are powered by one or more rechargeable batteries 114. In the illustrated embodiment, the batteries 114 are USB rechargeable 18650 cell batteries contained within the interior area formed by the top shell 104. EVA space fillers J can be used to contain and retain the batteries. Other embodiments can use other battery or power cell configurations. The batteries are coupled to the circuit board with wires that extend through apertures in the center disk F and upper structure disk C. The circuit board can be connected to a USB port 116, such as a sub USB port that allows for connection to a compatible USB charger unit or other external power source to provide recharging power to the batteries and/or data communication with the circuit board 112. The circuit board 112 and batteries can also be coupled to lights, such as status lights, integrated into the bottom and/or top shell 102/104 and visible from the exterior of the housing to provide status information to a user. The rechargeable batteries 114 allow the unit to be powered for 6+ hours at 50% volume—and recharged via the integrated USB port 116.

As seen in FIG. 3, the top shell has a contoured central receptacle portion 120 that contains a push button switch H, and the switch is covered by a flexible molded logo patch I retained in the central receptacle portion 120. A user can press on the molded logo patch I to activate the push button switch H, so as to activate the transducer 108, indicator lights, and/or components on the circuit board 112. In the illustrated embodiment, the circuit board 112 is a printed circuit board (PCB) above and isolated from the transducer 108. The PCB includes the controller, amplifier, on/off switch, a headphone jack could be provided in the housing and coupled to the PCB. The headphones are an accessory unit that allow the user to audibly listen to the program frequencies.

The circuit board 112 is configured to communicate with a remote unit, such as a phone, tablet, personal computer or the like, via an app that delivers selected programs or tracks for operation of the transducer 108 at selected frequencies, amplitudes (i.e., volume) and durations for one or more treatment tracks, patterns, or protocols. The App can include or provide access to a library of tracks or programs to control the transducer for the selected vibro-acoustic therapy treatment or series of treatments. The App can be configured so only the tracks or programs on the App or accessed via the App can be used to deliver the program or track for controlling the transducer 108. The App and the circuit board 112 can be configured to allow for pre-calibration of the devices, for example, through sonic reflection of audio signals. Programs, such as from one or more accessible libraries, will be available via in-app purchases. Accordingly, the vibro-acoustic assembly 100 can be used to deliver very specifically targeted and focused treatment to the particular area of the patient's body by selecting the desired programs or tracks from the library.

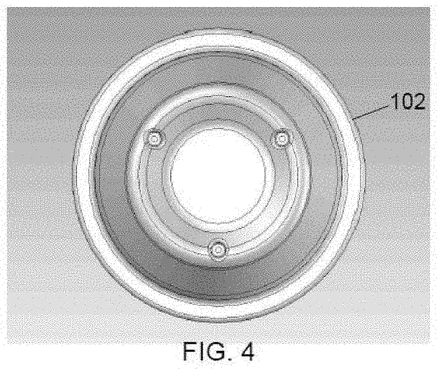

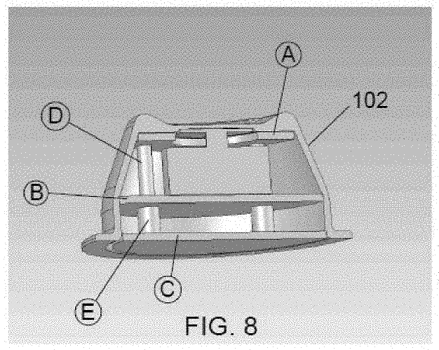

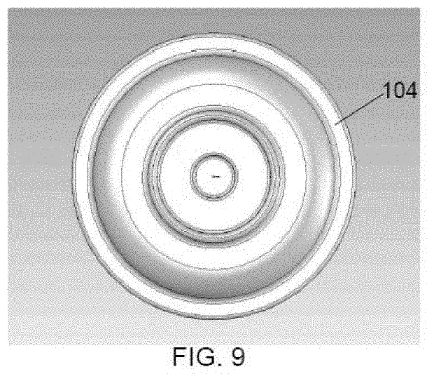

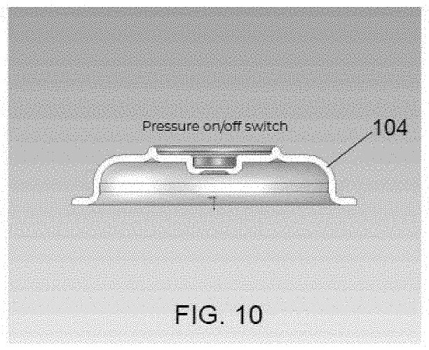

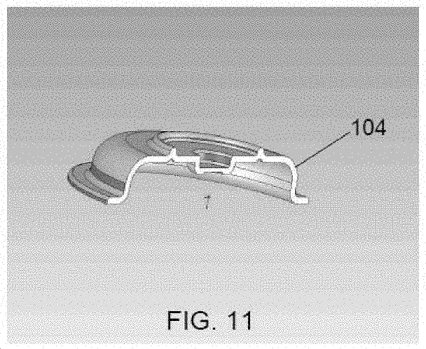

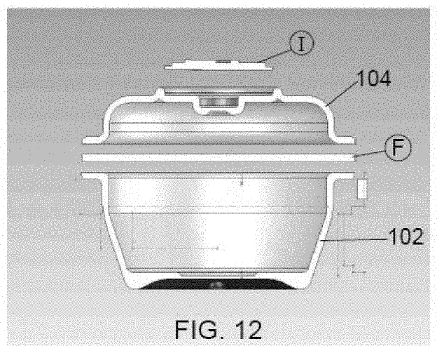

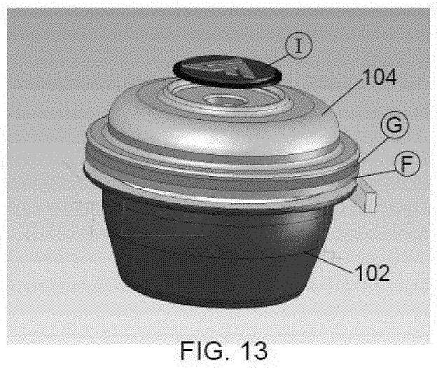

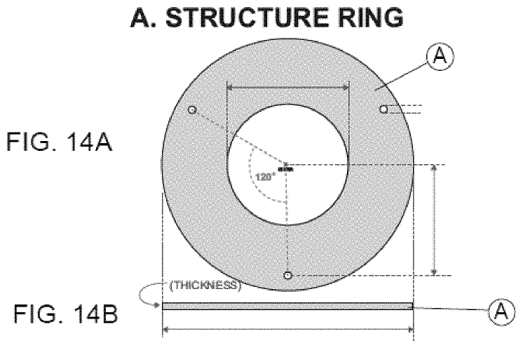

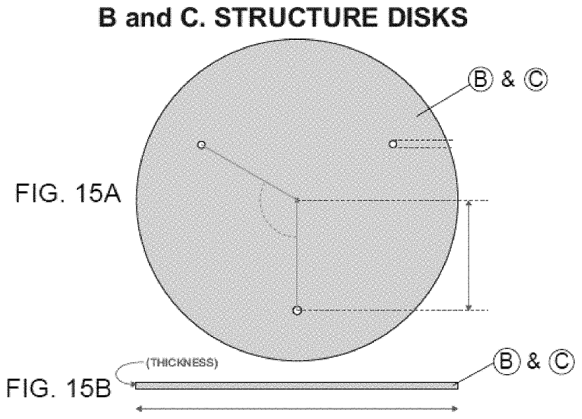

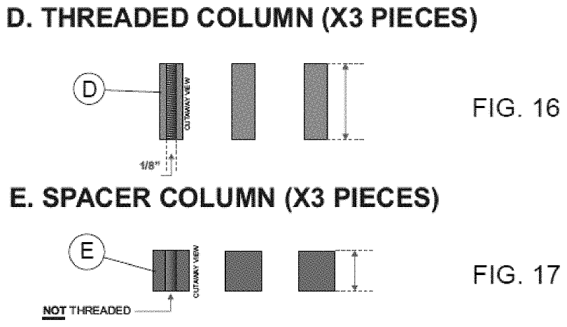

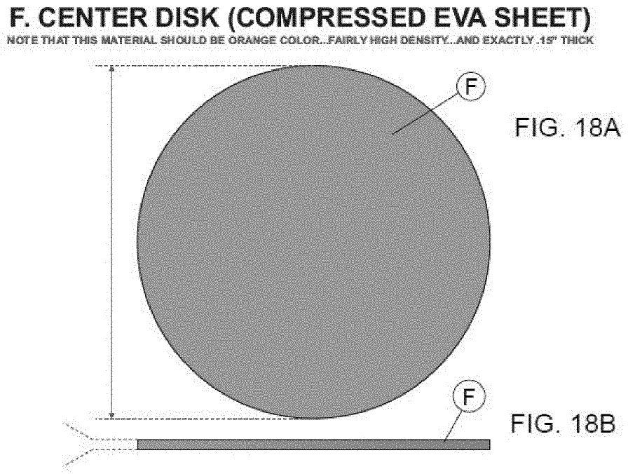

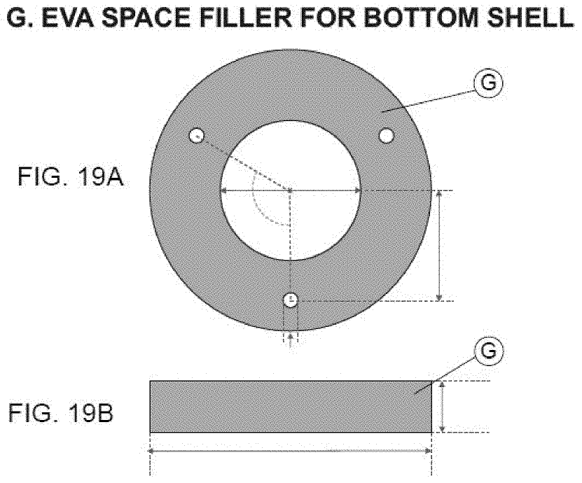

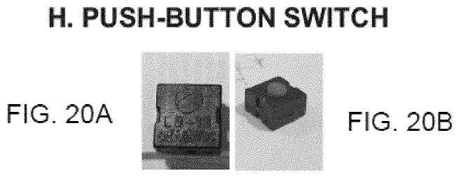

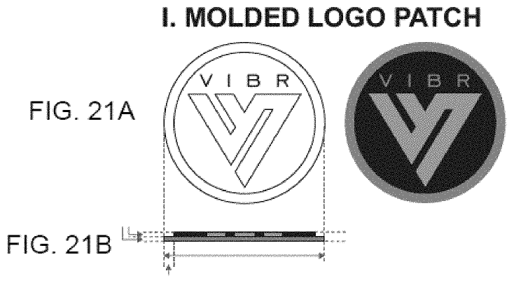

FIGS. 4-8 illustrate an embodiment of the bottom shell 102 with the inner structural frame formed by structure ring A (see also FIGS. 14A & 14B), the intermediate and upper structure disks B and C (see also FIGS. 15A & 15B), the threaded columns D (see also FIG. 16), and the spacer columns E (see also FIGS. 17). FIGS. 9-11 illustrate and embodiment of the upper shell 104, and FIGS. 12 and 13 illustrate an embodiment of the housing with the bottom and top shells 104 and 102, the center disk F (see also FIGS. 18A & 18B) and space filler G (see also FIGS. 19A & 19B), and the molded logo patch I (see also FIGS. 21A & 21B). An embodiment of the push button switch H is illustrated in FIGS. 20A & 20B, although other switch mechanisms could be used to turn the vibro-acoustic assembly 100 on and off, and/or between selected modes. The internal components, such as the transducer 108, the circuit board 112, the batteries 114, etc., fit within the housing's interior area 106, wherein the internal frame structure and the top and bottom shells 102 and 104 provide structure and stability.

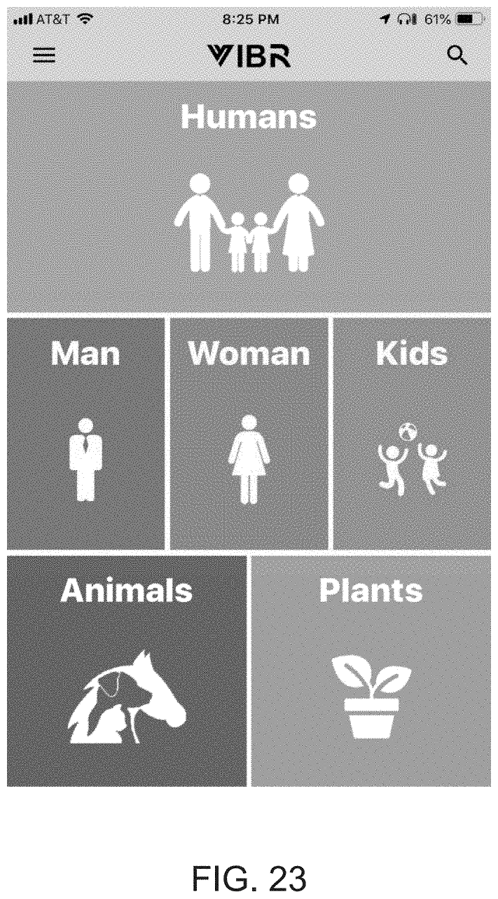

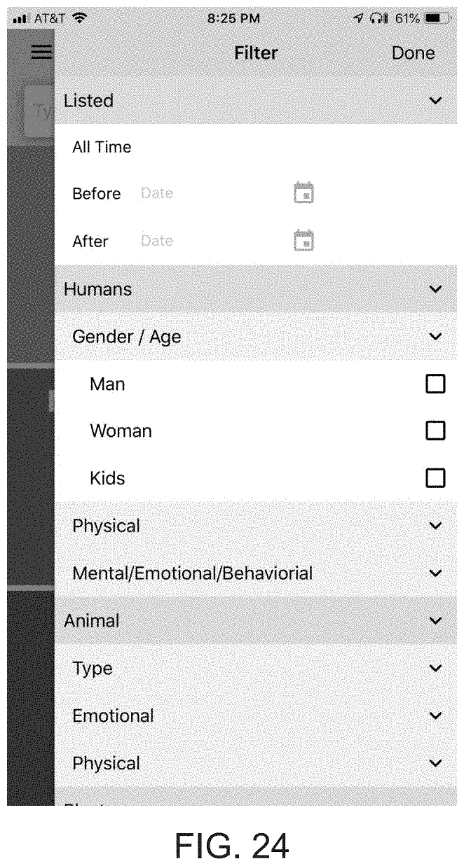

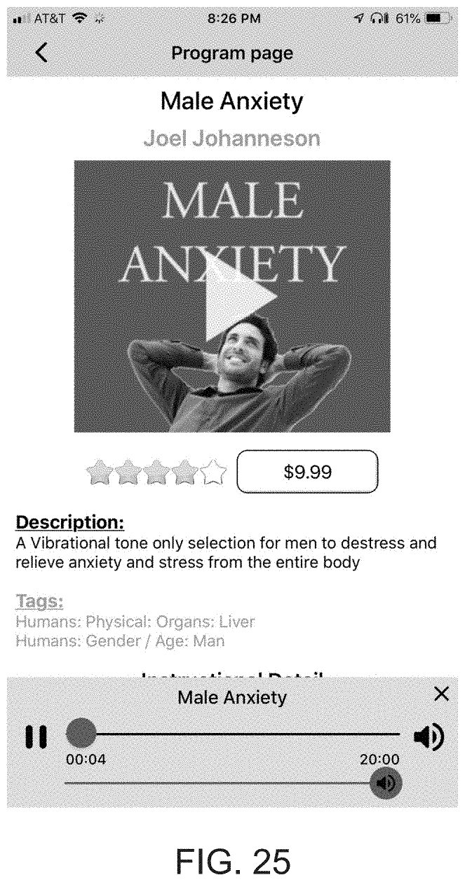

In operation of the vibro-acoustic assembly 100, a user launches the App via a smartphone, tablet, laptop, etc., and the App pairs exclusively with the available vibro-acoustic assemblies 100. FIGS. 23-25 are screenshots from the App as launched on a smartphone or tablet. The App can also control synchronization and calibration of multiple vibro-acoustic assemblies 100 that will be used together for a selected treatment program. A conventional Bluetooth pairing protocol is used. The App and/or the user will provide a visual and/or auditory indication that the R and/or L vibro-acoustic assemblies 100 are paired with the App. Once connected, the user selects a “program” or track from the App, such as via a library on or available via the App. The App is also configured to set and control the amplitude (volume) and frequency to provide the selected vibration intensity. In the illustrated embodiment, the vibration intensity is adjusted by a master volume control on the App. This controls the volume on the one or more paired and synchronized vibro-acoustic assemblies 100. The user then starts to play the track via the App, applies the vibro-acoustic assembly 100 to the selected portion of the patient's body and begins the session.

The App and/or the controller can control the session, including the frequency, volume, and duration, and the App and controller will terminate the session at the end of the track or in the event an intermediate event occurs requiring termination of the session, such as insufficient power reserves in the batteries to complete the session. In some embodiments, after the user selects a track, the App and the controller can query the batteries to determine whether they contain a sufficient charge to perform a full session of the selected track and associated session. If insufficient reserve power is available, the App can provide an error signal to the user or instructions to select a different track. When the track ends and the session is over, the user pushes the molded logo panel to press the button and turn off the vibro-acoustic assembly 100.

In some embodiments, the vibro-acoustic assembly 100 can include additional bio-feedback components, such as one or more temperature sensors, blood oxygen sensors, or other sensors that allow the controller in the vibro-acoustic assembly 100 to monitor body conditions to provide bio-feedback for control of the transducer and/or controller. For example, if a temperature sensor indicates that the temperature of the targeted area of the patient's body increased past a threshold or is changing at an unacceptable rate, the controller can turn off the transducer to stop treatment. Other bio-feedback components can be used and coupled to the controller and/or the transducer.

From the foregoing, it will be appreciated that specific embodiments of the invention have been described herein for purposes of illustration, but that various modifications may be made without deviating from the spirit and scope of the invention. Additionally, aspects of the invention described in the context of particular embodiments or examples may be combined or eliminated in other embodiments. Although advantages associated with certain embodiments of the invention have been described in the context of those embodiments, other embodiments may also exhibit such advantages. Additionally, not all embodiments need necessarily exhibit such advantages to fall within the scope of the invention. Accordingly, the invention is not limited except as by the appended claims.

I/We claim:

1. A hand-held vibro-acoustic assembly, comprising:
A housing comprising a top portion coupled to a bottom portion to define an interior area, the bottom portion having an engaging area configured to engage a body portion of a user;
one or more batteries in the interior area;
an activatable transducer in the interior area and operatively connected to the one or more batteries, the transducer being controllable and operable to generate vibrations in the range of approximately 15 Hz-10,000 Hz and transmittable through the engaging area of the housing's bottom portion to the body portion of the user, wherein the transducer is wirelessly controllable by an external controller and controllable for generation of vibrations in accordance with a selected one of a plurality of treatment protocols;
a structure ring in the interior area and coupled to the bottom portion of the housing, the structure ring having an open central area, and at least a portion of the transducer is positioned in the open central area;
an upper structure disk in the interior area and positioned approximately proximate to a top portion of the housings bottom portion;
an intermediate structure disk in the interior area and positioned between and spaced apart from each of the structure ring and the upper structure disk;
a plurality of support columns extending between the structure ring and the intermediate structure disk and forming a space that contains the transducer;
a plurality of spacer columns extending between the intermediate structure disk and the upper structure disk; and
a circuit board assembly positioned between the upper structure disk and the intermediate structure disk and comprising an amplifier, Bluetooth, battery charger, indicator, processors, and control features coupled to the transducer and connected to a USB port,
an activation switch connected to the upper portion of the housing and coupled to the transducer and the circuit board to activate the transducer to generate the acoustic vibrations for transmission to the body portion of the user.
